(19) United States
(12) Patent Application Publication
(10) Pub. No.: US 2023/0210877 A1
(43) Pub. Date: Jul. 6, 2023

(54) A Composition for Management of COVID-19 and Associated Disorders

(71) Applicant: Vedicinals India Private Limited, Talawade, Pune, Maharashtra (IN)

(72) Inventors: Prakash Punklik Salunke, Talawade, Pune, Maharashtra (IN); Vaisahli Prakash Salunke, Talawade, Pune, Maharashtra (IN); Pravin Ekanath Patil, Aurangabad, Maharashtra (IN)

(21) Appl. No.: 17/927,801

(22) PCT Filed: May 29, 2021
(86) PCT No.: PCT/IB2021/054727

§ 371 Date: Nov. 25, 2022

(30) Foreign Application Priority Data
May 29, 2020 (IN) 202021022638
Apr. 19, 2021 (IN) 202121017933

(51) Int. Cl. A61K 31/7048; A61K 31/12; A61K 31/353; A61K 31/4525; A61K 36/752; A61K 36/9066; A61K 36/82; A61K 36/489; A61K 36/48; A61K 36/185; A61K 36/67; A61K 9/00; A61K 9/10; A61P 31/14

(52) U.S. Cl. CPC A61K 31/7048; A61K 31/12; A61K 31/353; A61K 31/4525; A61K 36/752; A61K 36/9066; A61K 36/82; A61K 36/489; A61K 36/48; A61K 36/185; A61K 36/67; A61K 9/0053; A61K 9/10; A61P 31/14

(57) ABSTRACT

The present disclosure provides a composition that may find utility in management of COVID-19. The inventors of the present disclosure surprisingly observed that ingredients of the composition of the present disclosure exhibit functional synergy therebetween, wherein the composition prevents virus replication and/or Virus entry into human cells, either wholly or in part, while modulating immune response of the patient. Hence, the composition of the present disclosure holds potential for wide spread usage in management of COVID-19, either alone or in combination with other preventive or palliative/symptomatic or therapeutic strategies.

### CROSS REFERENCE TO RELATED APPLICATIONS

This application is the U.S. national stage of International Application No. PCT/IB2021/054727, filed on 2021 May 29. The international application claims the priority of IN 202021022638 filed on 2020 May 29 and the priority of IN 202121017933 filed on 2021 Apr. 19; all applications are incorporated by reference herein in their entirety.

### BACKGROUND

### Field of the Invention

The present disclosure relates generally to the field of pharmaceutical compositions. Particularly, the present disclosure provides a composition for management of COVID-19 and associated disorders.

### Background of the Invention

Background description includes information that may be useful in understanding the present invention. It is not an admission that any of the information provided herein is prior art or relevant to the presently claimed invention, or that any publication specifically or implicitly referenced is prior art.

The new mutated strain of Corona virus (CoV) was first identified in Wuhan, China, at the end of 2019 and initially it was named as 2019-nCoV [J. Microbiol. Immunol. Infect, pp. 1-3, 2020] The Emergency Committee of the World Health Organization (WHO) confirmed an outbreak in China on Jan. 30, 2020, which was, and still is a Public Health Emergency of International Concern [Travel Med. Infect. Dis., vol. 33, 2020] Officially, WHO named this CoV COVID-19 (coronavirus disease 2019), on Feb. 11, 2020 [H O Bull., no. JANUARY, pp. 1-7, 2020] COVID-19 is primarily spreading between people during close contact, often via small droplets, produced during coughing, sneezing or talking of an infected person. These droplets remain in air for few hours, infectious over a large distance or it may fall on the ground or other surfaces. People become infected by touching these contaminated surfaces and then their faces. It is most contagious during the first three days after onset of symptoms, although spread may be possible even before symptoms may appear and in later stages of the disease as well. The time from exposure to onset of symptoms is typically around five days, but may range from two to fourteen days.

Coronaviruses (CoVs) causes intense infections in humans as well as animals, which can lead to severe disorder in multiple organs including but not limited to respiratory tract, digestive tract and also systemically. Common symptoms of a person infected with coronavirus include respiratory symptoms, fever, cough, shortness of breath, and dyspnea. In more severe cases, infection can cause pneumonia, severe acute respiratory syndrome (SARS), renal failure, and even fatal consequences [Vet. Q., vol. 40, no. 1, pp. 1-12] Currently, no known and proven or tested therapies or treatment for COVID-19 exist till the date, but worldwide a lot of resources are diverted on finding a cure. Presently, the approach remains limited to preventive and symptomatic supportive therapies, in an attempt to prevent further complications and organ failure. Recommended preventive measures include hand washing, covering mouth while coughing, maintaining distance from other people, and monitoring and self-isolation for people who suspect they are infected. Government and authorities worldwide have responded by implementing travel restrictions, quarantines, curfews, workplace hazard controls, and facility closures which is causing severe impact on the world economy. In the fight against coronavirus, scientists have come up with three broad strategies for developing new drugs [Nat Rev Drug Discov 2016; 15:327-47] The first strategy is to test existing broad-spectrum anti-virals [J Infect 2013; 67:606-16] The advantages of these therapies are that their metabolic characteristics, dosages used, potential efficacy and side effects are clear as they have been approved for treating viral infections. But the disadvantage is that these therapies are too “broad-spectrum” and cannot kill coronaviruses in a targeted manner, and their side effects cannot be underestimated. The second strategy is to use existing molecular databases to screen for molecules that may have therapeutic effect on [Antimicrob Agents Chemother 2014; 14:4875-84 & Antimicrob Agents Chemother 2014; 58:4885-93] High-throughput screening makes this strategy possible, and new functions of many drug molecules can be found through this strategy, for example, the discovery of anti-HIV infection drug lopinavir/ritonavir. The third strategy is based on the genomic information and pathological characteristics of different coronaviruses to develop new targeted drugs from scratch. Theoretically, the drugs found through these strategies would exhibit better anti-coronavirus effects, but the research procedure of new drug might take several years, or even more than 10 years [Lancet Infect Dis 2014; 14:1090-5]

There is, therefore, a need in the art to develop a composition that may find utility in management of COVID-19 and associated disorders.

### OBJECTS OF THE INVENTION

Primary object of the present invention is to provide a composition that may aid in management of COVID-19 and associated disorders.

Another object of the invention is to provide a composition for enhancing immunity and general well-being of a subject.

Another object of the invention is to provide a composition that is substantially devoid of any side effects.

Another object of the invention is to provide a composition that is easy to prepare and economical.

Other objects of the present invention will be apparent from the description of the invention herein below.

### SUMMARY

The present disclosure relates generally to the field of pharmaceutical compositions. Particularly, the present disclosure provides a composition that may find utility in management of COVID-19 and associated disorders.

The inventors of the present disclosure surprisingly observed that ingredients of the composition of the present disclosure exhibit functional synergy therebetween, wherein the composition prevents virus replication and/or virus entry into human cells, either wholly or in part, while modulating immune response of the patient; the composition further has organ/cell protective properties, affords regeneration of cells, aids in suppressing bacterial co-infection and affords prevention of post-COVID-19 complications. Accordingly, the composition of the present disclosure can find utility in management of COVID-19 and associated disorders, either alone or in combination with other preventive or palliative/symptomatic or therapeutic strategies.

An aspect of the present disclosure provides a composition for management of COVID-19 and associated disorders, said composition comprising any or a combination of a therapeutically effective amount of Citrus reticulate, a therapeutically effective amount of Curcuma longa, a therapeutically effective amount of Camellia sinensis, a therapeutically effective amount of Sophora japonica L., a therapeutically effective amount of Arachis hypogaea, a therapeutically effective amount of Oroxylum indicum, and a therapeutically effective amount of Piper nigrum L. In an embodiment, the composition of the present disclosure may find utility in management of COVID-19.

In an embodiment, the composition comprises a therapeutically effective amount of Citrus reticulate, a therapeutically effective amount of Curcuma longa, a therapeutically effective amount of Camellia sinensi, a therapeutically effective amount of Sophora japonica L., a therapeutically effective amount of Arachis hypogaea, a therapeutically effective amount of Oroxylum indicum, and a therapeutically effective amount of Piper nigrum L. In an embodiment, the composition further comprises one or more excipients.

In an embodiment, the composition comprises: (i) active ingredients including: Citrus reticulata in an amount ranging from about 2% to about 20% by weight of the active ingredients, preferably from about 5% to about 10% by weight of the active ingredients; Curcuma longa in an amount ranging from about 5% to about 20% by weight of the active ingredients, preferably from about 10% to about 15% by weight of the active ingredients; Camellia sinensis in an amount ranging from about 5% to about 25% by weight of the active ingredients, preferably about 10% to about 20% by weight of the active ingredients; Sophora japonica L. in an amount ranging from about 10% to about 40% by weight of the active ingredients, preferably from about 20% to about 30% by weight of the active ingredients; Arachis hypogaea in an amount ranging from about 2% to about 20% by weight of the active ingredients, preferably from about 5% to about 15% by weight of the active ingredients; Oroxylum indicum in an amount ranging from about 20% to about 40% by weight of the active ingredients, preferably from about 25% to about 40% by weight of the active ingredients; and Piper nigrum L. in an amount ranging from about 0.01% to about 3.0% by weight of the active ingredients, preferably from about 0.03% to about 2.0% by weight of the active ingredients; and (ii) one or more excipients. In an embodiment, the composition comprises active ingredients in an amount ranging from 0.5% to about 80% by weight of the composition.

In an embodiment, the composition comprises: (i) active ingredients including: an extract of Citrus reticulata in an amount ranging from about 2% to about 20% by weight of the active ingredients, preferably from about 5% to about 10% by weight of the active ingredients; an extract of Curcuma longa in an amount ranging from about 5% to about 20% by weight of the active ingredients, preferably from about 10% to about 15% by weight of the active ingredients; an extract of Camellia sinensis in an amount ranging from about 5% to about 25% by weight of the active ingredients, preferably about 10% to about 20% by weight of the active ingredients; an extract of Sophora japonica L. in an amount ranging from about 10% to about 40% by weight of the active ingredients, preferably from about 20% to about 30% by weight of the active ingredients; an extract of Arachis hypogaea in an amount ranging from about 2% to about 20% by weight of the active ingredients, preferably from about 5% to about 15% by weight of the active ingredients; an extract of Oroxylum indicum in an amount ranging from about 20% to about 40% by weight of the active ingredients, preferably from about 25% to about 40% by weight of the active ingredients; and an extract of Piper nigrum L. in an amount ranging from about 0.01% to about 3.0% by weight of the active ingredients, preferably from about 0.03% to about 2.0% by weight of the active ingredients; and (ii) one or more excipients.

In an embodiment, the composition includes each of Citrus reticulate, Curcuma longa, Camellia sinensis, Sophora japonica L., Arachis hypogaea, Oroxylum indicum, and Piper nigrum L. in an amount such that the composition includes: Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Piperine in an amount ranging from about 0.03% to about 3% by weight of the active constituents.

In an embodiment, the composition for management of COVID-19 and associated disorders includes: active constituents including Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Piperine in an amount ranging from about 0.03% to about 3% by weight of the active constituents. In an embodiment, the composition further includes one or more excipients.

In an embodiment, the composition is formulated into a liquid formulation. In an embodiment, the composition is formulated into an orally ingestible suspension.

Other aspects, advantages, and salient features of the invention will become apparent to those skilled in the art from the following detailed description, which, taken in conjunction with the exemplary embodiments of the invention.

### DETAILED DESCRIPTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein. The examples used herein are intended merely to facilitate an understanding of the ways in which the embodiments herein may be practiced and to further enable those of skill in the art to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The present disclosure relates generally to the field of pharmaceutical compositions. Particularly, the present disclosure provides a composition that may find utility in management of COVID-19 and associated disorders.

The term “management of COVID-19 and associated disorders” as used herein, throughout the present disclosure, denotes diseases or disorders associated with infection from SARS-CoV-2 virus and strains/variants thereof, including fatigue, shortness of breath or difficulty in breathing, cough, joint pain, chest pain, memory, concentration or sleep problems, muscle pain, headache, fast or pounding heartbeat, loss of smell or taste, depression or anxiety, fever, dizziness, blood clotting, myocardial infarction, lung damage, severe acute respiratory syndrome (SARS), renal failure, inflammation, strokes, seizures, Guillain-Barre syndrome and such other disorders as known to or appreciated by persons skilled in the art.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non-limiting, i.e., other steps and other ingredients which do not affect the end of result can be added. The above terms encompass the terms “consisting of” and “consisting essentially of”.

As used herein, the terms “composition”, “blend”, or “mixture” are all intended to be used interchangeably.

The terms “weight percent”, “vol-%”, “percent by weight”, “% by weight”, and variations thereof, as used herein, refer to the concentration of a substance as the weight of that substance divided by the total weight of the composition and multiplied by 100. It is understood that, as used here, “percent”, “%”, and the like are intended to be synonymous with “weight percent”, “vol-%”, etc.

In some embodiments, the numbers expressing quantities of ingredients, properties such as concentration, reaction conditions, and so forth, used to describe and claim certain embodiments of the invention are to be understood as being modified in some instances by the term “about”. Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

An aspect of the present disclosure provides a composition for management of COVID-19 and associated disorders, said composition comprising any or a combination of a therapeutically effective amount of Citrus reticulata, a therapeutically effective amount of Curcuma longa, a therapeutically effective amount of Camellia sinensis, a therapeutically effective amount of Sophora japonica L., a therapeutically effective amount of Arachis hypogaea, a therapeutically effective amount of Oroxylum indicum, and a therapeutically effective amount of Piper nigrum L. In an embodiment, the composition of the present disclosure may find utility in management of COVID-19 and associated disorders.

In an embodiment, the composition comprises a therapeutically effective amount of Citrus reticulata, a therapeutically effective amount of Curcuma longa, a therapeutically effective amount of Camellia sinensis, a therapeutically effective amount of Sophora japonica L, a therapeutically effective amount of Arachis hypogaea, a therapeutically effective amount of Oroxylum indicum, and a therapeutically effective amount of Piper nigrum L. In an embodiment, the composition further comprises one or more excipients.

In an embodiment, Citrus reticulata comprises peels of Citrus reticulate. In an embodiment, Citrus reticulate contains citrus bioflavonoids in an amount of not less than 40%. In an embodiment, Citrus reticulata contains hesperidin in an amount of not less than 35%. In an embodiment, Citrus reticulate comprises powdered peels of Citrus reticulata in powdered form. In an embodiment, Citrus reticulata comprises extract of Citrus reticulate that contains citrus bioflavonoids in an amount of not less than 40%. In an embodiment, the extract is in powdered form.

In an embodiment, Curcuma longa comprises rhizomes of Curcuma longa. In an embodiment, rhizomes of Curcuma longa are in powdered form.

In an embodiment, Curcuma longa comprises extract of Curcuma longa, preferably of rhizomes of Curcuma longa. In an embodiment, Curcuma longa comprises ethyl acetate extract of Curcuma longa, preferably of rhizomes of Curcuma longa. In an embodiment, the extract is in powdered form. In an embodiment, the extract contains curcuminoids in an amount of not less than 60% w/w. In an embodiment, the extract contains bisdem ethoxy curcumin in an amount ranging from about 2.2% w/w to about 6.5%. In an embodiment, the extract contains desmethoxycurcumin in an amount ranging from about 10.0% to about 19.0%. In an embodiment, the extract contains curcumin in an amount ranging from about 45.0% to about 85.0%.

In an embodiment, Camellia sinensis comprises leaves of Camellia sinensis. In an embodiment, leaves of Camellia sinensis are in powdered form.

In an embodiment, Camellia sinensis comprises extract of Camellia sinensis, preferably of leaves of Camellia sinensis. In an embodiment, Camellia sinensis comprises hydro alcoholic extract of Camellia sinensis, preferably of leaves of Camellia sinensis. In an embodiment, Camellia sinensis comprises extract of Camellia sinensis, preferably of leaves of Camellia sinensis. In an embodiment, the extract contains caffeine in an amount of not more than about 7.0% w/w. In an embodiment, the extract contains epigallocatechin gallate (EGCG) in an amount of not less than about 30.0% w/w. In an embodiment, the extract contains total catechins in an amount of not less than about 55.0% w/w. In an embodiment, the extract contains total polyphenols in an amount ranging from about 60.0% w/w to about 96% w/w. In an embodiment, the extract contains epigallocatechin in an amount ranging from about 8.0% w/w to about 17.0% w/w. In an embodiment, the extract contains epicatechin in an amount ranging from about 2.0% w/w to about 9.0% w/w. In an embodiment, the extract contains epicatechin gallate in an amount ranging from about 8.0% w/w to about 17.0% w/w. In an embodiment, the extract contains catechin in an amount ranging from about 0.5% w/w to about 3.5% w/w. In an embodiment, the extract contains gallocatechin gallate in an amount ranging from about 5.0% w/w to about 12.0% w/w.

In an embodiment, Sophora japonica L. comprises flowers of Sophora japonica L. In an embodiment, flowers of Sophora japonica L. are in powdered form.

In an embodiment, Sophora japonica L. comprises extract of Sophora japonica L., preferably, of flowers of Sophora japonica l . . . . In an embodiment, Sophora japonica L. comprises hydroalcoholic extract of Sophora japonica L., preferably of flowers of Sophora japonica L. In an embodiment, the extract contains quercetin in an amount of not less than about 30% w/w. In an embodiment, the extract contains rutin in an amount of not less than about 30% w/w. In an embodiment, the extract is in powdered form.

In an embodiment, Arachis hypogaea comprise extract of pod shells of Arachis hypogaea. In an embodiment, Arachis hypogaea comprises alcoholic extract of pod shells of Arachis hypogaea. In an embodiment, the extract of pod shells of Arachis hypogaea is in powdered form. In an embodiment, the extract of pod shells of Arachis hypogaea contains luteolin in an amount not less than about 17% w/w on dry basis.

In an embodiment, Oroxylum indicum comprises bark of Oroxylum indicum. In an embodiment, the bark of Oroxylum indicum is in powdered form. In an embodiment, the powdered bark of Oroxylum indicum contains Oroxylin A in an amount of not less than about 5.0% w/w and Baicalin in an amount of not less than about 6.0% w/w.

In an embodiment, Oroxylum indicum comprises extract of Oroxylum indicum, preferably of bark of Oroxylum indicum. In an embodiment, Oroxylum indicum comprises alcoholic extract of Oroxylum indicum, preferably of bark of Oroxylum indicum. In an embodiment, the extract is in powdered form. In an embodiment, the extract contains Oroxylin A in an amount of not less than about 5.0% w/w. In an embodiment, the extract contains Baicalin in an amount of not less than about 6.0% w/w.

In an embodiment, Piper nigrum L. comprises fruits of Piper nigrum L. In an embodiment, the fruits of Piper nigrum L. are in powdered form. In an embodiment, Piper nigrum L. comprises extract of fruits of Piper nigrum L. In an embodiment, Piper nigrum L. comprises hydroalcoholic extract of fruits of Piper nigrum L. In an embodiment, the extract is in powdered form. In an embodiment, the extract contains pipeline in an amount of not less than about 45% w/w.

In an embodiment, the composition for management of COVID-19 and associated disorders includes: (i) active ingredients including: Citrus reticulata in an amount ranging from about 2% to about 20% by weight of the active ingredients, preferably from about 5% to about 10% by weight of the active ingredients; Curcuma longa in an amount ranging from about 5% to about 20% by weight of the active ingredients, preferably from about 10% to about 15% by weight of the active ingredients; Camellia sinensis in an amount ranging from about 5% to about 25% by weight of the active ingredients, preferably about 10% to about 20% by weight of the active ingredients; Sophora japonica L. in an amount ranging from about 10% to about 40% by weight of the active ingredients, preferably from about 20% to about 30% by weight of the active ingredients; Arachis hypogaea in an amount ranging from about 2% to about 20% by weight of the active ingredients, preferably from about 5% to about 15% by weight of the active ingredients; Oroxylum indicum in an amount ranging from about 20% to about 40% by weight of the active ingredients, preferably from about 25% to about 40% by weight of the active ingredients; and Piper nigrum L. in an amount ranging from about 0.01% to about 3.0% by weight of the active ingredients, preferably from about 0.03% to about 2.0% by weight of the active ingredients; and (ii) one or more excipients. In an embodiment, the composition comprises active ingredients in an amount ranging from 0.5% to about 80% by weight of the composition, preferably, in an amount ranging from 1% to about 60% by weight of the composition, and more preferably, in an amount ranging from 3% to about 50% by weight of the composition. In an embodiment, the composition further includes one or more excipients.

In an embodiment, the composition for management of COVID-19 and associated disorders comprises: (i) active ingredients including: an extract of Citrus reticulate in an amount ranging from about 2% to about 20% by weight of the active ingredients, preferably from about 5% to about 10% by weight of the active ingredients; an extract of Curcuma longa in an amount ranging from about 5% to about 20% by weight of the active ingredients, preferably from about 10% to about 15% by weight of the active ingredients; an extract of Camellia sinensis in an amount ranging from about 5% to about 25% by weight of the active ingredients, preferably about 10% to about 20% by weight of the active ingredients; an extract of Sophora japonica L in an amount ranging from about 10% to about 40% by weight of the active ingredients, preferably from about 20% to about 30% by weight of the active ingredients; an extract of Arachis hypogaea in an amount ranging from about 2% to about 20% by weight of the active ingredients, preferably from about 5% to about 15% by weight of the active ingredients; an extract of Oroxylum indicum in an amount ranging from about 20% to about 40% by weight of the active ingredients, preferably from about 25% to about 40% by weight of the active ingredients; an extract of Piper nigrum L. in an amount ranging from about 0.01% to about 3.0% by weight of the active ingredients, preferably from about 0.03% to about 2.0% by weight of the active ingredients; and (ii) one or more excipients. In an embodiment, the composition comprises active ingredients in an amount ranging from 0.5% to about 80% by weight of the composition, preferably, in an amount ranging from 1% to about 60% by weight of the composition, and more preferably, in an amount ranging from 3% to about 50% by weight of the composition.

In an embodiment, the composition includes each of Citrus reticulate, Curcuma longa, Camellia sinensis, Sophora japonica L., Arachis hypogaea, Oroxylum indicum, and Piper nigrum L. in an amount such that the composition includes: Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Piperine in an amount ranging from about 0.03% to about 3% by weight of the active constituents.

Table 1 below illustrates active ingredients of the composition and active constituent(s) against each of the active ingredients. It should be appreciated that the advantageous composition of the present disclosure may be realized, either using the active ingredients (or extract thereof) or by directly using the active constituents thereof or by using combinations thereof. Alike active ingredients and extracts thereof, active constituents are also available commercially and the same may be used to realize the advantageous composition of the present disclosure.

TABLE 1
Details of the Composition

| Active | | Active | |
|---|---|---|---|
| Ingredients | Amount (%) | Constituents | Amount (%) |
| Citrus reticu [symbol] | 2 to 20 | Hesperidin | 10 to 25 |
| Curcuma longa | 5 to 20 | Curcumin | 15 to 35 |
| Camellia sinensis | [symbol] | Epigallocat [symbol] | 15 to 30 |
| Sophora japonica | 10 to 40 | Rutin | 10 to 25 |
| | | Quercetin | 0.5 to 8.0 |
| Arachis hypogaea | 2 to 20 | Luteolin | 1 to 10 |
| Orox [symbol] indicum | 20 to 40 | Baicalin | 1 to 15 |
| Piper nigrum [symbol] | 0.01 to 3.0 | Piperine | 0.03 to 3.0 |

[symbol]

indicates data missing or illegible when filed

In an embodiment, the composition includes: active constituents including Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Piperine in an amount ranging from about 0.03% to about 3% by weight of the active constituents.

In an embodiment, the composition is formulated into a liquid formulation. In an embodiment, the composition is formulated into an orally ingestible suspension.

Another aspect of the present disclosure provides a suspension formulation, said suspension comprising, (i) active constituents comprising: a therapeutically effective amount of Hesperidin; a therapeutically effective amount of Curcumin; a therapeutically effective amount of Epigallocatechin; a therapeutically effective amount of Rutin; a therapeutically effective amount of Quercetin; a therapeutically effective amount of Luteolin; a therapeutically effective amount of Baicalin; and a therapeutically effective amount of Piperine; and (ii) one of more excipients.

In an embodiment, the suspension formulation comprises: (i) active constituents comprising: Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Pipeline in an amount ranging from about 0.03% to about 3% by weight of the active constituents; and (ii) one or more excipients. In an embodiment, the suspension formulation comprises active constituents in an amount ranging from about 1% to about 20% by weight of the composition and the rest being one or more excipients.

In an embodiment, the suspension formulation comprises: (i) active constituents in an amount ranging from about 3% to about 10% by weight of the composition, said active constituents comprising: Hesperidin in an amount of about 16.22% by weight of the active constituents; Curcumin in an amount of about 26.06% by weight of the active constituents; Epigallocatechin in an amount of about 22.28% by weight of the active constituents; Rutin in an amount of about 18.45% by weight of the active constituents; Quercetin in an amount of about 2.68% by weight of the active constituents; Luteolin in an amount of about 5.14% by weight of the active constituents; Baicalin in an amount of about 8.83% by weight of the active constituents; and Piperine in an amount of about 0.34% by weight of the active constituents; and (ii) the rest being one or more excipients.

In an embodiment, the suspension formulation comprises, in 50 ml of the suspension, Hesperidin in an amount ranging from 500 mg to 850 mg, preferably, 600 mg to 750 mg, Curcumin in an amount ranging from 800 mg to 1250 mg, preferably, 900 mg to 1200 mg, Epigallocatechin in an amount ranging from 600 mg to 1050 mg, preferably, 750 to 1000 mg, Rutin in an amount ranging from 600 mg to 900 mg, preferably, 650 mg to 850 mg, Quercetin in an amount ranging from 50 mg to 200 mg, preferably, 75 mg to 125 mg, Luteolin in an amount ranging from 100 mg to 300 mg, preferably, 150 mg to 250 mg, Baicalin in an amount ranging from 125 mg to 450 mg, preferably, 150 to 400 mg, Piperine in an amount ranging from 5 mg to 50 mg, preferably, 10 mg to 30 mg, and glycyrrhizin in an amount ranging from 300 mg to 700 mg, preferably, 400 mg to 600 mg.

In an embodiment, the composition is formulated into a lozenge. In an embodiment, the composition is formulated into an orally dispersible tablet. In an embodiment, the composition is formulated into an orally ingestible liquid. In an embodiment, the composition is formulated into syrup. In an embodiment, the composition is formulated into an orally ingestible suspension. In an embodiment, the composition is formulated into a nasal drop. In an embodiment, the composition is formulated as a liquid ready for inhalation or aerosolization. In an embodiment, the composition is formulated as a liquid ready for nebulization. In an embodiment, the composition is formulated into an injectable solution. However, it should be appreciated that the instant composition can be formulated into any liquid, semi-solid or solid formulation in accordance with the desired route of administration.

Another aspect of the present disclosure relates to a method of management of COVID-19 and associated disorders in a subject, said method comprising administering to the subject in need thereof an effective amount of a composition, said composition comprising: (i) active constituents including Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Pipeline in an amount ranging from about 0.03% to about 3% by weight of the active constituents; and (ii) one or more excipients.

Further aspect of the present disclosure relates to a composition for use in management of COVID-19 and associated disorders, said composition comprising: (i) active constituents including Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Pipeline in an amount ranging from about 0.03% to about 3% by weight of the active constituents; and (ii) one or more excipients.

Still another aspect of the present disclosure relates to use of a composition for manufacture of a medicament for management of COVID-19 and associated disorders, said composition comprising: (i) active constituents including Hesperidin in an amount ranging from about 10% to about 25% by weight of the active constituents; Curcumin in an amount ranging from about 15% to about 35% by weight of the active constituents; Epigallocatechin in an amount ranging from about 15% to about 30% by weight of the active constituents; Rutin in an amount ranging from about 10% to about 25% by weight of the active constituents; Quercetin in an amount ranging from about 0.5% to about 8% by weight of the active constituents; Luteolin in an amount ranging from about 1% to about 10% by weight of the active constituents; Baicalin in an amount ranging from about 1% to about 15% by weight of the active constituents; and Pipeline in an amount ranging from about 0.03% to about 3% by weight of the active constituents; and (ii) one or more excipients.

Another aspect of the present disclosure relates to a method of management of COVID-19 and associated disorders in a subject, said method comprising administering to the subject in need thereof an effective amount of a suspension formulation, said suspension comprises, in 50 ml of the suspension, Hesperidin in an amount ranging from 500 mg to 850 mg, preferably, 600 mg to 750 mg, Curcumin in an amount ranging from 800 mg to 1250 mg, preferably, 900 mg to 1200 mg, Epigallocatechin in an amount ranging from 600 mg to 1050 mg, preferably, 750 to 1000 mg, Rutin in an amount ranging from 600 mg to 900 mg, preferably, 650 mg to 850 mg, Quercetin in an amount ranging from 50 mg to 200 mg, preferably, 75 mg to 125 mg, Luteolin in an amount ranging from 100 mg to 300 mg, preferably, 150 mg to 250 mg, Baicalin in an amount ranging from 125 mg to 450 mg, preferably, 150 to 400 mg, Pipeline in an amount ranging from 5 mg to 50 mg, preferably, 10 mg to 30 mg, and glycyrrhizin in an amount ranging from 300 mg to 700 mg, preferably, 400 mg to 600 mg.

Further aspect of the present disclosure relates to a suspension formulation for use in management of COVID-19 and associated disorders, said suspension comprises, in 50 ml of the suspension, Hesperidin in an amount ranging from 500 mg to 850 mg, preferably, 600 mg to 750 mg, Curcumin in an amount ranging from 800 mg to 1250 mg, preferably, 900 mg to 1200 mg, Epigallocatechin in an amount ranging from 600 mg to 1050 mg, preferably, 750 to 1000 mg, Rutin in an amount ranging from 600 mg to 900 mg, preferably, 650 mg to 850 mg, Quercetin in an amount ranging from 50 mg to 200 mg, preferably, 75 mg to 125 mg, Luteolin in an amount ranging from 100 mg to 300 mg, preferably, 150 mg to 250 mg, Baicalin in an amount ranging from 125 mg to 450 mg, preferably, 150 to 400 mg, Piperine in an amount ranging from 5 mg to 50 mg, preferably, 10 mg to 30 mg, and glycyrrhizin in an amount ranging from 300 mg to 700 mg, preferably, 400 mg to 600 mg.

Another aspect of the present disclosure provides a tablet, said tablet comprising a therapeutically effective amount of Citrus reticulata, a therapeutically effective amount of Curcuma longa, a therapeutically effective amount of Camellia sinensis, a therapeutically effective amount of Sophora japonica L, a therapeutically effective amount of Arachis hypogaea, a therapeutically effective amount of Oroxylum indicum, and a therapeutically effective amount of Piper nigrum L. In an embodiment, the composition further comprises one or more excipients.

In an embodiment, the one or more excipients includes any or a combination of a bulking agent, a solubilizer, a binder, a disintegrant, a chelating agent, a lubricant, a thickening agent, a glidant, a flavouring agent, a colouring agent, a tonicity agent, a sweetening agent, a buffering agent, a preservative, a suspending agent and a solvent.

In an embodiment, bulking agent (s) include but not limited to, lactose USP, Starch 1500, mannitol, sorbitol, maltodextrin, maltitol or other non-reducing sugars; microcrystalline cellulose (e.g., Avicel), dibasic calcium phosphate (anhydrous or dihydrate), sucrose, etc. and mixtures thereof. However, a person skilled in the art would appreciate that any other bulking agent(s) can be utilized to serve the intended purpose without departing from the scope and spirit of the invention.

In an embodiment, solubilizer(s) includes but not limited to, cyclodextrins, pH adjusters, salts and buffers, surfactants, fatty acids, phospholipids, metals of fatty acids and the likes. However, a person skilled in the art would appreciate that any other solubilizing agent(s) can be utilized to serve the intended purpose without departing from the scope and spirit of the invention.

In an embodiment, binder(s) include but not limited to, cellulosic derivatives (such as methylcellulose, carboxymethyl cellulose, hydroxyethyl cellulose, hydroxyethyl methyl cellulose, etc), polyacrylates (such as Carbopol, polycarbophil, etc), Povidone (all grades), Polyox of any molecular weight or grade, irradiated or not, starch, polyvinylpyrrolidone (PVP), Avicel, and the like. However, a person skilled in the art would appreciate that any other binder(s) can be utilized to serve the intended purpose without departing from the scope and spirit of the invention.

In an embodiment, glidant(s) includes but not limited to, colloidal silicon dioxide, precipitated silicon dioxide, fumed silica (CAB-O-SIL M-5P, trademark of Cabot Corporation), stearowet and sterotex, silicas (such as SILOID and SILOX silicas—trademarks of Grace Davison Products, Aerosil—trademark of Degussa Pharma), higher fatty acids, the metal salts thereof, hydrogenated vegetable oils and the like. However, a person skilled in the art would appreciate that any other glidant(s) can be utilized to serve the intended purpose without departing from the scope and spirit of the invention.

In an embodiment, flavoring agent (s) includes but not limited to, fruit aromas such as orange, banana, strawberry, cherry, wild cherry, lemon; cardamom, anis, mint, menthol, vanillin, and ethyl vanillin, and other similar aromas, or the mixtures thereof. However, a person skilled in the art would appreciate that any other flavoring agent(s) can be utilized to serve the intended purpose without departing from the scope and spirit of the invention.

In an embodiment, sweetener(s) includes but not limited to, sucralose, acesulfame-K, aspartame, saccharine or saccharine sodium and calcium salts, sodium cyclamate, sucrose, fructose, glucose, sorbitol, Glycyrrhizin or the mixtures thereof. In an embodiment, the sweetener includes Glycyrrhizin. However, a person skilled in the art would appreciate that any other sweetener(s) can be utilized to serve the intended purpose without departing from the scope and spirit of the invention.

In an embodiment, buffer system includes but not limited to, sodium citrate, potassium citrate, sodium citrate di-hydrate, citric acid, citric acid monohydrate, sodium bicarbonate, potassium bicarbonate, sodium di-hydrogen phosphate and potassium di hydrogen phosphate and the likes or combination thereof. However, a person skilled in the art would appreciate that any other buffer system can be utilized to serve the intended purpose without departing from the scope and spirit of the invention.

In an embodiment, solvents includes but not limited to, methanol, ethanol, n-propanol, isopropanol, hexane, heptane, petroleum ether, cyclohexane, diethyl ether, disopropyl ether, ethyl acetate, methyl acetate, ethyl formate, methyl formate, isobutyl acetate, n-butyl acetate, methylene chloride, ethylene chloride, chloroform, carbon tetrachloride, acetone, ethyl methyl ketone, diisobutyl ketone, methyl isobutyl ketone, 1,4-dioxane, toluene, ammonia solution, glacial acetic acid, ammonium hydroxide, sodium hydroxide, calcium hydroxide, calcium carbonate, potassium hydroxide, potassium carbonate, water and the likes. However, a person skilled in the art would appreciate that any other solvent or a combination of solvents can be utilized to serve the intended purpose without departing from the scope and spirit of the invention.

In an embodiment, the suspending includes but not limited to, anionic surfactants such as ammonium lauryl sulfate, sodium lauryl sulfate sodium laureth sulfate (sodium lauryl ether sulfate or SLES), and sodium myreth sulfate, Docusate (dioctyl sodium sulfosuccinate), Perfluorooctanesulfonate (PFOS), Perfluorobutanesulfonate, Alkyl-aryl ether phosphates, Alkyl ether phosphates, sodium stearate, sodium lauroyl sarcosinate and carboxylate-based fluoro surfactants such as perfluorononanoate, perfluorooctanoate (PFOA or PFO), cationic surfactants such as quaternary ammonium salts like cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB), non-ionic surfactants such as Fatty alcohol ethoxylates, Alkylphenol ethoxylates, Fatty acid ethoxylates and the likes, Fatty acid esters of sorbitol like Sorbitan monolaurate, Sorbitan monostearate, Sorbitan tristearate, Tween, Fatty acid esters of glycerol like Glycerol monostearate and Glycerol monolaurate, polyelectrolytes, wetting agents, and stabilizing agents.

Although various embodiments of the present disclosure are detailed herein referencing to parts of the herbs and/or to extracts of the herbs (or of parts thereof), either in the powdered form or otherwise, it is to be appreciated that the same (or one or more active constituents thereof) may be used either in its/their micronized form or in conjugated form (e.g. conjugation or linking of hesperidin with alpha glucosyl moiety) or such other forms to improve its solubility and/or bioavailability. A person skilled in the art is expected to be acquiesced with the approaches routinely used for improving solubility and/or bioavailability of ingredients to meet the formulation requirements, and the same are not detailed herein for the sake of brevity. Accordingly, in one, few or all of the embodiments of the present disclosure, the composition may comprise active constituents of one, few or all of the herbs mentioned therein, instead of usage of herbs or parts thereof or usage of extract of herbs or of parts thereof. Further, one, few or all of the active constituent(s), present in the composition, may be in micronized form, conjugated form or such other forms that improves or otherwise aids in improving solubility or bioavailability thereof to achieve the desired formulation characteristics.

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIGS. 1A and 1B illustrate snippets depicting the effect of the developed composition on histopathology of hearts of rats suffering from myocardical infarction as compared to the Control group FIG. 2 illustrates an exemplary graph depicting the effect of SV9 (S+ V9) and S on patient's report turning negative in days (RT-PCR) in COVID-19 positive patients from day 0 to 14, in accordance with the embodiments of the present disclosure.

FIG. 3 illustrates an exemplary graph depicting the effect of SV9 (S+ V9) and S on CT value (Viral Load) of COVID-19 positive patients from day 0 to 5, in accordance with the embodiments of the present disclosure.

FIG. 4 illustrates an exemplary graph depicting the effect of SV9 (S+ V9) and S on serum IL-6 levels (pg/mL) of COVID-19 positive patients from day 0 to 5 and 0 to 12, in accordance with the embodiments of the present disclosure.

FIG. 5 illustrates an exemplary graph depicting the effect of SV9 (S+ V9) and S on serum CRP levels (mg/L) of COVID-19 positive patients from day 0 to 5 and 0 to 12, in accordance with the embodiments of the present disclosure.

FIG. 6 illustrates an exemplary graph depicting the effect of SV9 (S+ V9) and S on serum Total antibody levels (mg/L) of COVID-19 positive patients from day 0 to 5 and 0 to 12, in accordance with the embodiments of the present disclosure.

FIG. 7 illustrates an exemplary graph depicting the effect of SV9 (S+ V9) and S on serum CPK levels (U/L) of COVID-19 positive patients from day 0 to 5 and 0 to 12, in accordance with the embodiments of the present disclosure.

FIG. 8 illustrates an exemplary graph depicting the effect of SV9 (S+ V9) and S on serum D-Dimer levels (pg FEU/L) of COVID-19 positive patients from day 0 to 12, in accordance with the embodiments of the present disclosure.

FIG. 9 illustrates an exemplary graph depicting the effect of SV9 (S+ V9) and S on serum ferritin levels (pg/L) of COVID-19 positive patients from day 0 to 12, in accordance with the embodiments of the present disclosure.

FIG. 10 illustrates an exemplary graph depicting the effect of SV9 (S+ V9) and S on serum CD4 levels (cells/pL) of COVID-19 positive patients from day 0 to 12, in accordance with the embodiments of the present disclosure.

FIG. 11 illustrates an exemplary graph depicting the effect of SV9 (S+ V9) and S on serum CD8 levels (cells/pL) of COVID-19 positive patients from day 0 to 12, in accordance with the embodiments of the present disclosure.

FIG. 12 illustrates an exemplary graph depicting the effect of SV9 (S+ V9) and S on serum CD19 levels (cells/pL) of COVID-19 positive patients from day 0 to 12, in accordance with the embodiments of the present disclosure.

FIG. 13 illustrates an exemplary graph depicting the effect of SV9 (S+ V9) and S on serum CD16/56 levels (cells/pL) of COVID-19 positive patients from day 0 to 12, in accordance with the embodiments of the present disclosure.

FIG. 14 illustrates an exemplary graph depicting the effect of SV9 (S+ V9) and S on COVID-19 patients at risk (staying positive) from day 0 to 14, in accordance with the embodiments of the present disclosure.

FIG. 15 illustrates an exemplary graph depicting the effect of SV9 (S+ V9) and S on cumulative no. of COVID-19 patients turning negative, in accordance with the embodiments of the present disclosure.

FIG. 16 illustrates a graph depicting the effect of SV9 (S+ V9) on patients (n=62 in each group) suffering from COVID-19, showing the percentage of patients with normal and abnormal findings of X-ray from day 0 to 45 of COVID-19 infection, in accordance with the embodiments of the present disclosure.

### DETAILED DESCRIPTION OF THE PREFERRED EMBODIMENTS

### Examples

### Preliminary Study

Coronavirus disease (COVID-19) has emerged as a pandemic and a public health crisis of global proportions. Most people who fell sick with COVID-19 experiences mild to moderate symptoms and recover without any special treatment. However, certain disorders have been associated with patients experiencing moderate to severe symptoms. Predominantly, such disorders associated with COVID-19 includes cytokine storm, acute lung inflammation, myocardial injury such as myocardial infarction, blood thickening and/or clot formation, impairment of brain function, breathlessness, body, joint and muscle pain and the likes, myocardial injury being the most notable posing a serious health problem. Keeping that in view, preliminary studies were conducted to understand the effects of Epigallocatechin gallate (EGCG) alone, Baicalin alone, Quercetin alone and when given as a combination, in an isoproterenol induced myocardial infarction rat model. Inventors of the present disclosure surprisingly observed that EGCG, Baicalin and Quercetin when given in combination, exhibits functional synergy therebetween, wherein the combination significantly reversed the parameters associated with myocardial infarction when compared to the individual treatments.

Preliminary study was carried out on total 60 rats that were equally divided into five groups: Group 1, Group 2, Group 3, Group 4 and Group 5. Group 1 represents the rats treated with the Isoproterenol at a dose of 90 mg/kg body weight (i.e. Control group); Group 2 represents the rats treated with—Isoproterenol 90 mg/kg body weight+Epigallocatechin gallate (EGCG) 138 mg/kg body weight; Group 3 represents the rats treated with—Isoproterenol 90 mg/kg body weight+Baicalin 138 mg/kg body weight; Group 4 represents the rats treated with—Isoproterenol 90 mg/kg body weight+Quercetin 138 mg/kg body weight; and Group 5 represents the rats treated with—Isoproterenol 90 mg/kg body weight+a combination containing EGCG 91.4 mg/kg body weight, Baicalin 36.3 mg/kg body weight and Quercetin 10.3 mg/kg body weight (a total of 138 mg/kg body weight).

Provided herein-below, in Table 2, are the results of the preliminary study establishing synergism between EGCG, Baicalin and Quercetin.

TABLE 2
Efficacy of compositions on isoproterenol
induced myocardial infarction rat model

| | PARAMETERS | | | |
|---|---|---|---|---|
| GROUPS | GPT | GOT | LDH | TNF-A |
| Group 1 | 92.83 | 109.17 | 188.33 | 577.56 |
| (Isoproterenol - 90 mg/kg bwt.) | | | | |
| Group 2 | 75.30 | 139.15 | [symbol] | 408.29 |
| (Isoproterenol - [symbol] mg/kg | | | | |
| bwt. + EGCG 138 mg/kg bwt.) | | | | |
| Group 3 | 79.57 | 110.37 | 138.67 | 438.58 |
| (Isoproterenol - 90 mg/kg | | | | |
| bwt. + Biacalin 138 mg/kg bwt.) | | | | |
| Group 4 | 90.47 | 103.33 | 121.00 | 411.70 |
| (Isoproterenol - 90 mg/kg | | | | |
| bwt. + [symbol] 138 mg/kg bwt.) | | | | |
| Group 5 | 74.17 | 97.43 | 119. [symbol] | 400.47 |
| (Isoproterenol - 90 mg/kg | | | | |
| bwt. + mixture 138 mg/kg bwt.) | | | | |

[symbol]

indicates data missing or illegible when filed

From Table 2, it is evident that EGCG, Baicalin and Quercetin when given in combination, they exhibits functional synergy therebetween, wherein the combination significantly reversed the parameters GPT, GOT, LDH and TNF-a associated with myocardial infarction when compared with individual treatments at the same dosage.

Based on the preliminary studies, Inventors of the present disclosure conducted multiple experiments including other active constituents/plant extracts as part of the above mixture. After series of experiments, inventors of the present disclosure surprisingly arrived at a composition that prevents virus replication and/or virus entry into human cells, either wholly or in part, while modulating immune response of the patient. It could further be observed that the composition of the present disclosure aids in providing relief from the COVID-19 associated disorders such as cytokine storm, acute lung inflammation, blood thickening (or clot formation), renal damage, muscle & joint pain, and myocardial injury such as myocardial infarction.

### Example 1—Orally Ingestible Suspension

### Formulation

50 ml orally ingestible suspension formulation was prepared, composition whereof is provided herein-below in Table 3:

TABLE 3
Suspension Formulation (V9)

| Active Constituents | Amount (in mg) |
|---|---|
| Hesperidin | 725 |
| Curcumin | 1165 |
| Epigallocatechin | 996 |
| Rutin | 825 |
| Quercetin | 120 |
| Lut [symbol] | 230 |
| Baicalin | 395 |
| Piperine | 15 |
| Glycyrrhizin | 529 |
| (sweetening agent) | |
| Excipients | q.s. to make 50 mL |

[symbol]

indicates data missing or illegible when filed

### Efficacy (Retention of Synergistic Activity)

Efficacy of the developed composition (composition shown in Table 3) was assessed on the isoproterenol induced myocardial infarction rat model to confirm retention of the synergistic activity.

TABLE 4
Efficacy of the composition on isoproterenol
induced myocardial infarction rat model

| | PARAMETERS | | | |
|---|---|---|---|---|
| GROUPS | GPT | GOT | LDH | TNF-A |
| Group 1 | 92.83 | 109.17 | 188.33 | 577.56 |
| (Isoproterenol - 90 mg/kg bwt.) | | | | |
| Group 2 | 74.53 | 94.47 | 123.67 | [symbol] |
| (Isoproterenol - 90 mg/kg | | | | |
| bwt. + V9 464 mg/kg bwt.) | | | | |

[symbol]

indicates data missing or illegible when filed

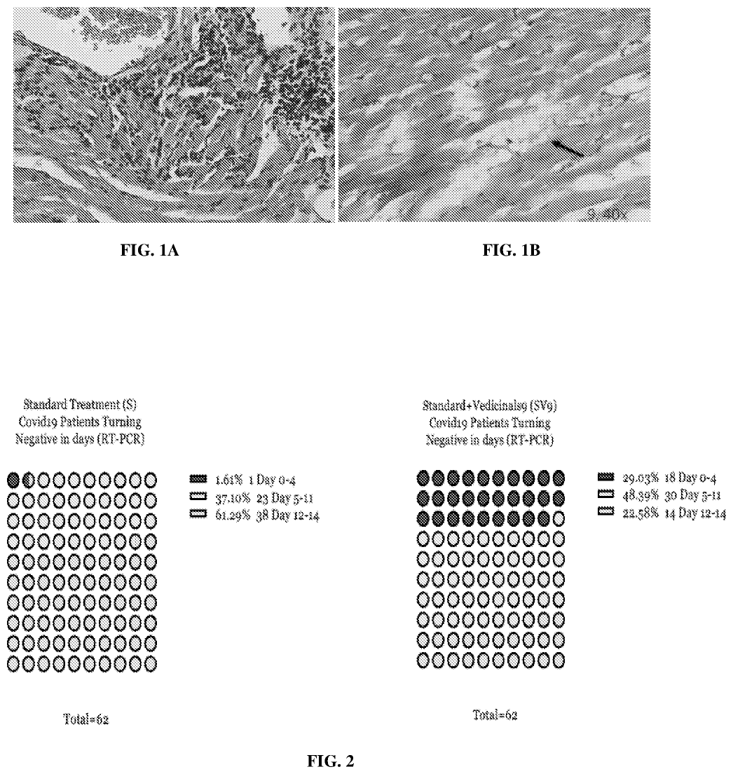

As can be seen from Table 4 above, the developed composition (as shown in Table 3 above) exhibits synergistic activity, comparable to (or even better than) the combination of EGCG, Baicalin and Quercetin, confirming that the presence of other active constituents does not hinder with the synergistic activity as regards reversal of the parameters GPT, GOT, LDH and TNF-a associated with/corresponding to the myocardial infarction. FIGS. 1A and 1B illustrate snippets depicting the effect of the developed composition (as shown in Table 3 above) on histopathology of hearts of rats suffering from myocardical infarction as compared to the Control group. Group 1 (FIG. 1A) rats showed myocardial degeneration, infiltration of inflammatory cells, and extra-vasated RBCs, whereas Group 2 (FIG. 1A) rats showed minimal myocardial degeneration, vacuolations, no infiltration of inflammatory cells or hemorrhages.

Clinical Trial

The suspension (V9) prepared in Example 1 above was subjected to clinical trial for management of COVID-19 and associated disorders. A randomized, open label, parallel efficacy, active control, multi-centre exploratory trial to evaluate efficacy and safety of the suspension (V9) as an adjunct treatment to standard treatment/standard intervention (hereinafter referred as S, provided herein below in Table 5) for the management of mild to moderate COVID-19 Patients. About 124 adults, exhibiting flu-like symptoms with confirmed COVID-19 RT-PCR test were selected for the study.

Study duration: Treatment—12±2 days and follow up—30 days.

Study Time-points: Screening (Day 2 to 0±2 days), Randomization & Hospitalization (Day 1 t 2 days), Day 5±2 days, Day 12 t 2 days and follow-up visit (Day 45±2 days)

Intervention: Patients were randomized to take either of the following drug regimens:

1) Standard intervention, S (n-62)

2) SV9 (Suspension formulation, V9+Standard intervention, S (n-62))

Dosage & administration: Standard intervention—as per the Ministry of Health and family welfare guidelines for COVID-19

Suspension formulation, V9 (5000 mg in 50 ml suspension per day)—Loading dose on day 1-25 ml each at 1 hour before breakfast, lunch and dinner; maintaining dose from day 2 to day 12±2 days—20 ml, 15 ml and 15 ml at 1 hour, before breakfast, lunch and dinner, respectively.

TABLE 5
Standard Care Treatment/Standard Intervention (S)

| Drug class or | | |
|---|---|---|
| Therapeutic | | |
| classification | Medicine name | Medicine name |
| Antibiotic | Doxycycline + [symbol] | [symbol] |
| | [symbol] | |
| | Cefixime | |
| [symbol] | [symbol] | — |
| Anti-pyretic | [symbol] | [symbol] |
| Nutritional Supplement | Vitamin C | Vitamin C |
| | [symbol] Vitamin | Calcium and Vitamin D3 |
| | C/Ascorbic Acid + L Lysine | |
| | Multivitamin including Zinc | Multivitamin |
| Steroid | [symbol] | [symbol] |
| | [symbol] | |
| Proton Pump | [symbol] | — |
| Inhibitor (PPI) | [symbol] | |
| Pro-biotic | | — |

[symbol]

indicates data missing or illegible when filed

FIG. 2 illustrates the effect of SV9 (S+ V9) on patient's report turning negative in days (RT-PCR) in COVID-19 positive patients from day 0 to 14 i.e. comparison of number of patients turning negative serum from day 0 to day 14, when V9 5000 mg adjuvant with standard intervention (S) compared with standard intervention alone (3). Data represented as change in expected and observed values in two comparable interventional group (n=62 per group).

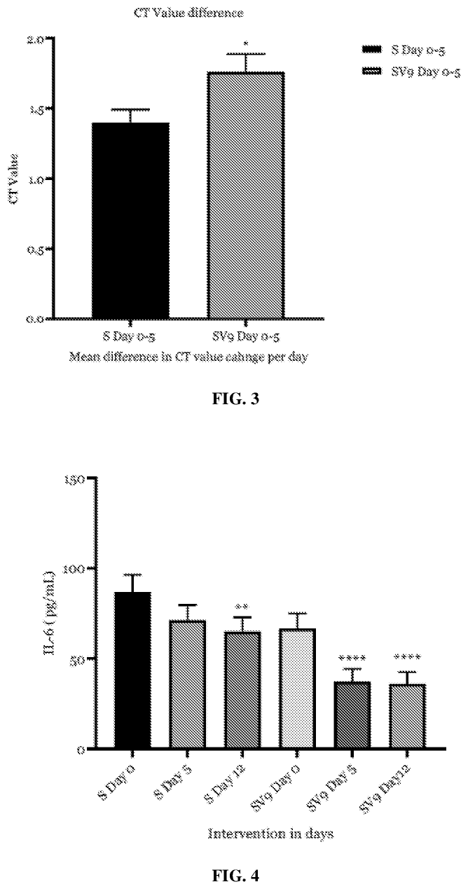

FIG. 3 illustrates the effect of SV9 (S+ V9) on CT value (Viral Load) of COVID-19 positive patients from day 0 to 5 i.e. comparison of mean difference in CT value per day (Viral Load) from day 0 to 5, when V9 5000 mg adjuvant with standard intervention (S) compared with standard intervention (S) alone. Data represented as change in CT value (Viral Load) levels (Mean±SEM) in two comparable interventional group (n=62 per group). Significant at *p<0.05, **p<0.01, *p<0.001, ****P<0.0001 when compared before and after intervention at day 5 both interventional groups.

FIG. 4 Illustrates the effect of SV9 (S+ V9) on serum IL-6 levels (pg/mL) of COVID-19 positive patients from day 0 to 5 and 0 to 12. Data interpreted as following: comparison of serum IL-6 levels (pg/mL) from day 0 to 5 and 0 to 12, when V9 5000 mg adjuvant with standard intervention (S) compared with standard intervention (S) alone. Data represented as change in serum IL-6 levels (Mean±SEM) in two comparable interventional group (n=62 per group). Significant at *p<0.05, **p<0.01, ***p<0.001, ****P<0.0001 when compared before and after intervention at day 5 and 12 in both interventional groups.

FIG. 5 Illustrates the effect of SV9 (S+ V9) on serum CRP levels (mg/L) of COVID-19 positive patients from day 0 to 5 and 0 to 12. Data interpreted as following: comparison of serum CRP levels (mg/L) from day 0 to 5 and 0 to 12, when V9 5000 mg adjuvant with standard intervention (S) compared with standard intervention (S) alone. Data represented as change in serum CRP levels (Mean±SEM) in two comparable interventional group (n=62 per group). Significant at *p<0.05, **p<0.01, ***p<0.001, ***P<0.0001 when compared before and after intervention at day 5 and 12 in both interventional groups.

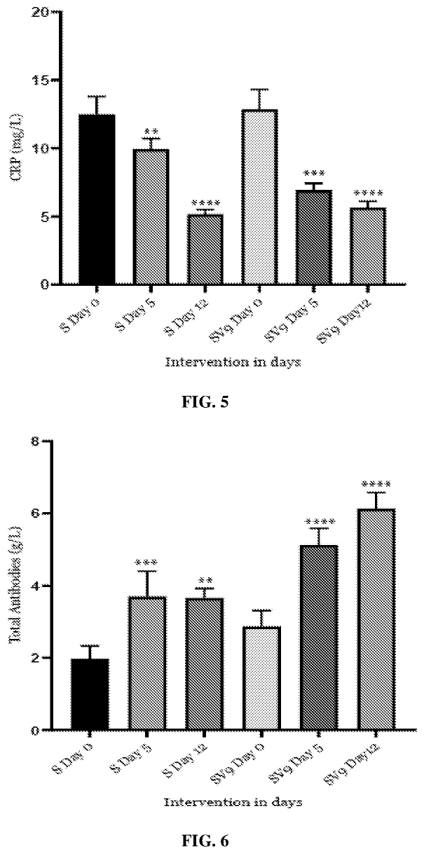

FIG. 6 Illustrates the effect of SV9 (S+ V9) on serum Total antibody levels (mg/L) of COVID-19 positive patients from day 0 to 5 and 0 to 12. Data interpreted as following: comparison of serum Total antibody levels (mg/L) from day 0 to 5 and 0 to 12, when V9 5000 mg adjuvant with standard intervention (S) compared with standard intervention (S) alone. Data represented as change in serum Total antibody levels (Mean±SEM) in two comparable interventional group (n=62 per group). Significant at *p<0.05, **p<0.01, ***p<0.001, ****p<0.0001 when compared before and after intervention at day 5 and 12 in both interventional groups.

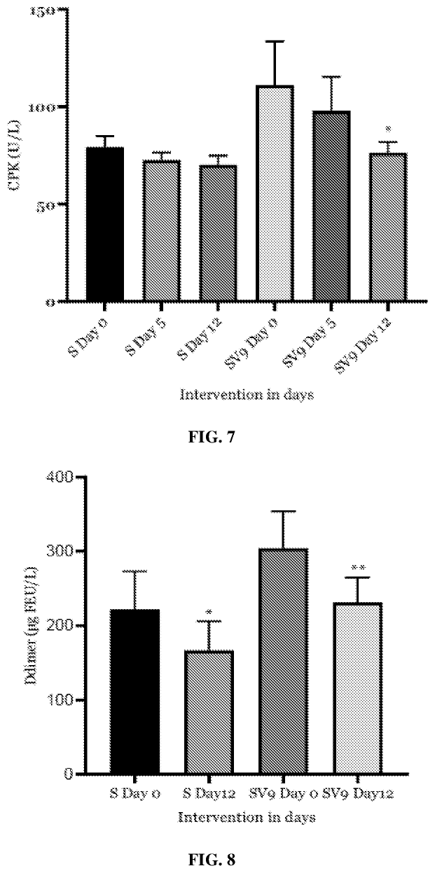

FIG. 7 illustrates the effect of SV9 (S+ V9) on serum CPK levels (U/L) of COVID-19 positive patients from day 0 to 5 and 0 to 12. Data interpreted as following: comparison of serum CPK levels (U/L) from day 0 to 5 and 0 to 12, when V9 5000 mg adjuvant with standard intervention (S) compared with standard intervention (S) alone. Data represented as change in serum CPK levels (Mean±SEM) in two comparable interventional group (n=62 per group). Significant at *p<0.05, when compared before and after intervention at day 5 and 12 in both interventional groups.

FIG. 8 illustrates the effect of SV9 (S+ V9) on serum D-Dimer levels (pg

FEU/L) of COVID-19 positive patients from day 0 to 12. Data interpreted as following: comparison of serum D-Dimer levels (pg FEU/L) from day 0 to day 12, when V9 5000 mg adjuvant with standard intervention (S) compared with standard intervention (S) alone. Data represented as change in serum D-Dimer levels (Mean±SEM) in two comparable interventional group (n=62 per group). Significant at *p<0.05, **p<0.01, ***p<0.001, ****p<00001, when compared before and after intervention at day 12 in both interventional groups.

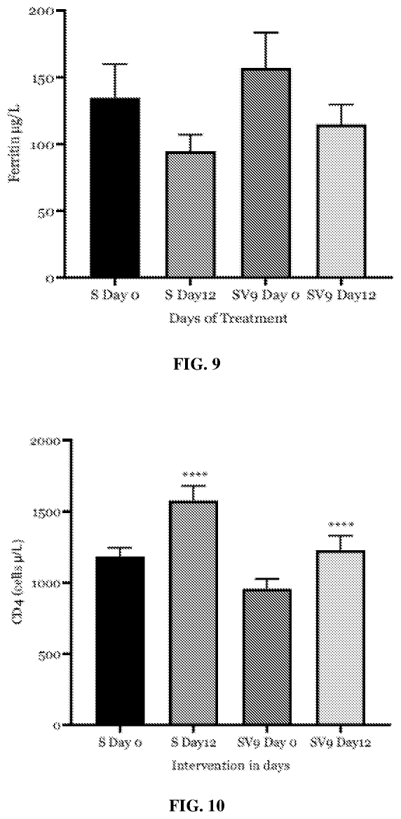

FIG. 9 illustrates the effect of SV9 (S+ V9) on serum ferritin levels (pg/L) of COVID-19 positive patients from day 0 to 12. Data interpreted as following: comparison of serum ferritin levels (pg/L) from day 0 to day 12, when V9 5000 mg adjuvant with standard intervention (S) compared with standard intervention (S) alone. Data represented as change in serum ferritin levels (Mean±SEM) in two comparable interventional group (n=62 per group).

FIG. 10 Illustrates the effect of SV9 (S+ V9) on serum CD4 levels (cells/pL) of COVID-19 positive patients from day 0 to 12. Data interpreted as following: comparison of serum CD4 levels (pg/L) from day 0 to day 12, when V9 5000 mg adjuvant with standard intervention (S) compared with standard intervention (S) alone. Data represented as change in serum CD4 levels (Mean±SEM) in two comparable interventional group (n=62 per group). Significant at *p<0.05, **p<0.01, ***p<0.001, ****P<0.0001, when compared before and after intervention at day 12 in both interventional groups.

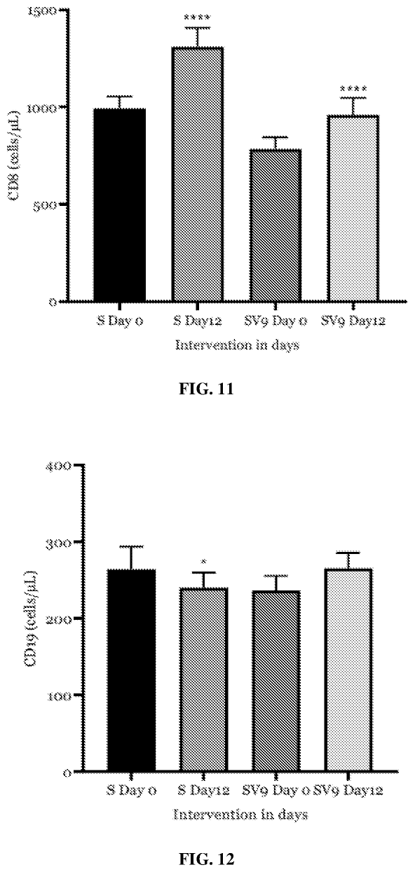

FIG. 11 Illustrates the effect of SV9 (S+ V9) on serum CD8 levels (cells/pL) of COVID-19 positive patients from day 0 to 12. Data interpreted as following: comparison of serum CD8 levels (pg/L) from day 0 to day 12, when V9 5000 mg adjuvant with standard intervention (S) compared with standard intervention (S) alone. Data represented as change in serum CD8 levels (Mean±SEM) in two comparable interventional group (n=62 per group). Significant at *p<0.05, **p<0.01, ***p<0.001, ****p<0.0001, when compared before and after intervention at day 12 in both interventional groups.

FIG. 12 Illustrates the effect of SV9 (S+ V9) on serum CD19 levels (cells/pL) of COVID-19 positive patients from day 0 to 12. Data interpreted as following: comparison of serum CD19 levels (pg/L) from day 0 to day 12, when V9 5000 mg adjuvant with standard intervention (S) compared with standard intervention (S) alone. Data represented as change in serum CD 19 levels (Mean±SEM) in two comparable interventional group (n=62 per group). Significant at *p<0.05, **p<0.01, ***p<0.001, ****P<0.0001, when compared before and after intervention at day 12 in both interventional groups.

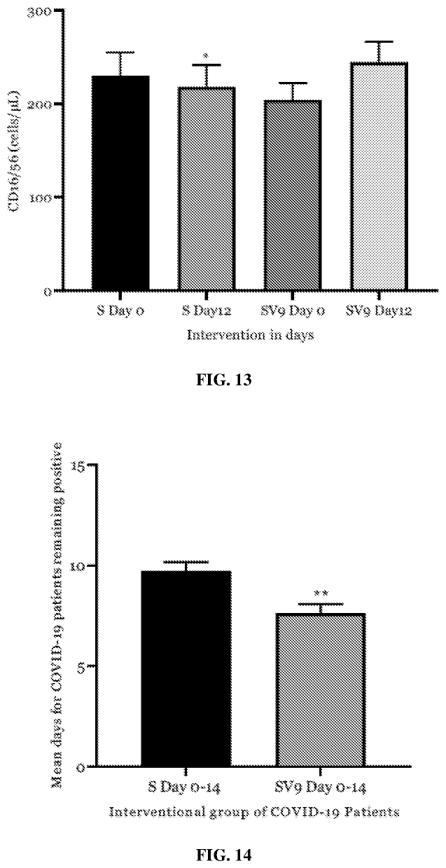

FIG. 13 Illustrates the effect of SV9 (S+ V9) on serum CD16/56 levels (cells/pL) of COVID-19 positive patients from day 0 to 12. Data interpreted as following: comparison of serum CD16/56 levels (pg/L) from day 0 to day 12, when V9 5000 mg adjuvant with standard intervention (S) compared with standard intervention (S) alone. Data represented as change in serum CD16/56 levels (Mean±SEM) in two comparable interventional group (n=62 per group). Significant at *p<0.05, **p<0.01, ***p<0.001, ****p<00001, when compared before and after intervention at day 12 in both interventional groups.

FIG. 14 Illustrates the effect of SV9 (S+ V9) on COVID-19 patients at risk (staying positive) from day 0 to 14. Data interpreted as following: comparison of mean difference in patients remaining positive from day 0 to 14, when V9 5000 mg adjuvant with standard intervention (S) compared with standard intervention (S) alone. Data represented as change in number of patients remaining positive (Mean±SEM) in two comparable interventional group (n=62 per group). Significant at *p<0.05, **p<0.01, ***p<0.001, ****p<00001 when compared before and after intervention at day 5 both interventional groups.

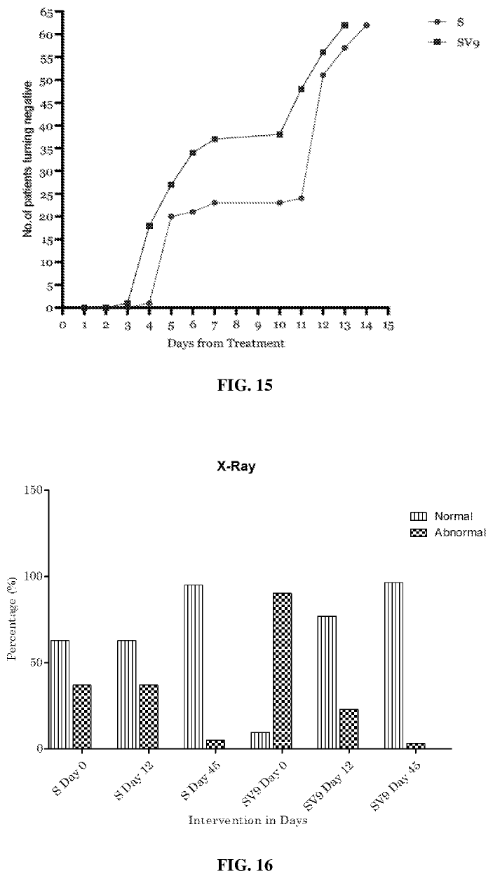

FIG. 15 Illustrates the effect of SV9 (S+ V9) and S on cumulative no. of COVID-19 patients turning negative.

FIG. 16 illustrates a graph depicting the effect of SV9 (S+ V9) on patients (n=62 in each group) suffering from COVID-19, showing the percentage of patients with normal and abnormal findings of X-ray from day 0 to 45 of COVID-19 infection. At day 0, 37.10% (n=23/62) of patients had abnormal X-ray findings in standard intervention (S) and 90.32% (n=56/62) in SV9 group. After 12±2 days of treatment with S and SV9, only 22.95% (n=14/61) of patients had abnormal findings in SV9 group, resulting in 77.05% (n=47/61) of patients with normal findings compared with standard intervention (S) that shows no change in patient conditions starting from day 0.

Table 6 below illustrates effect of the composition of the present disclosure on Prothrombin Time (PT).

TABLE 6
Effect of the composition on Prothrombin Time (PT)
PT (in seconds)

| | | | | | Mean ± SEM | | |
|---|---|---|---|---|---|---|---|
| | Mean ± SEM | | | | Day 0 vs | Day 0 vs | Day 0 vs |
| Intervention | Day 0 | Day 5 | Day 12 | Day 45 | Day 5 | Day 12 | Day 45 |
| Standard (S) | 16.61 ± 0.24 | 16. [symbol] 6 ± 0.26 | 16.48 ± 0.25 | 16.64 ± 0.34 | [symbol] | [symbol] | [symbol] |
| | | | | | (0.7638) | (0.4095) | (0.7747) |
| SV9 | 16.15 ± 0.27 | 15.9 [symbol] ± 0.48 | 15.7 [symbol] ± 0.35 | 1 [symbol] .66 ± 0.3 [symbol] | [symbol] | [symbol] | [symbol] |
| | | | | | (0.62 [symbol] 2) | (0.1388) | (0.1022) |

[symbol]

indicates data missing or illegible when filed

### Example 2: Orally Ingestible Suspension

50 ml orally ingestible suspension formulation was prepared, composition whereof is provided in Table 7 below.

TABLE 7
Composition for orally ingestible suspension

| Active Constituents | Amount (in mg) |
|---|---|
| [symbol] extract | 1052 |
| [symbol] extract | [symbol] |
| Rutin | [symbol] |
| [symbol] extract | [symbol] |
| [symbol] | [symbol] |
| [symbol] extract | 200 |
| [symbol] powder | 100 |
| Piper nigrum (as Piperine) extract | 15 |
| [symbol] | 500 |
| (sweetening agent) | |
| Other Excipients | q.s. to make 50 mL |

[symbol]

indicates data missing or illegible when filed

The proposed composition along with alleviating infected persons also supports in faster recovery by providing Immuno-modulatory, Neuro-protective, Anti-Inflammatory, Vital organ/tissue protective and Anti-oxidant effects. The proposed composition is with optimum nutrition, non-toxic, natural herbal plant extracts, easy to digest, have health protective and rejuvenate functions to ameliorate the symptoms and/or syndromes of COVID-19 and general health & well-being.

Accordingly, the present disclosure provides broad spectrum, highly effective and synergistic composition including one or more standardized Phyto-extracts from one or more plant components, providing a combination of target compounds and % thereof, chosen specifically considering genetics, various possibilities of infection, multiplication, and transmission of SARS-CoV-2, the type and amount of which are carefully calculated to provide therapeutically or prophylactically desired effect of inhibition of cellular entry, multiplication and transmission of SARS-CoV-2 and for management of disorders associated with COVID-19.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### ADVANTAGES

The present disclosure provides a composition that may aid in management of COVID-19 and associated disorders.

The present disclosure provides a composition for enhancing immunity and general well-being of a subject.

The present disclosure provides a composition that is substantially devoid of any side effects.

The present disclosure provides a composition that is easy to prepare and economical.

Other advantages of the present disclosure will be apparent from the description of the invention provided herein above.

1. A composition for management of COVID-19 and associated disorders, said composition comprising:
(i) active constituents comprising:
Hesperidin in an amount ranging from 10% to 25% by weight of the active constituents;
Curcumin in an amount ranging from 15% to 35% by weight of the active constituents;
Epigallocatechin in an amount ranging from 15% to 30% by weight of the active constituents;
Rutin in an amount ranging from 10% to 25% by weight of the active constituents;
Quercetin in an amount ranging from 0.5% to 8% by weight of the active constituents;
Luteolin in an amount ranging from 1% to 10% by weight of the active constituents;
Baicalin in an amount ranging from 1% to 15% by weight of the active constituents; and
Piperine in an amount ranging from 0.03% to 3% by weight of the active constituents; and
(ii) one or more excipients.

2. The composition as claimed in claim 1, wherein the composition comprises active constituents in an amount ranging from 1% to 20% by weight of the formulation and the rest being one or more excipients.

3. The composition as claimed in claim 1, wherein the excipient includes any or a combination of: a bulking agent, a solubilizer, a binder, a lubricant, a thickening agent, a flavouring agent, a colouring agent, a tonicity agent, a sweetening agent, a suspending agent, a buffering agent, a preservative, and a solvent.

4. The composition as claimed in claim 1, wherein the composition is formulated into a lozenge, a tablet, an ingestible liquid, a syrup, a suspension, a nasal drop, an injectable solution, an aerosol and a liquid ready for nebulization.

5. An orally ingestible suspension formulation for management of COVID-19 and associated disorders, said formulation comprising:
(i) active constituents comprising: Hesperidin in an amount ranging from 10% to 25% by weight of the active constituents;
Curcumin in an amount ranging from 15% to 35% by weight of the active constituents;
Epigallocatechin in an amount ranging from 15% to 30% by weight of the active constituents;
Rutin in an amount ranging from 10% to 25% by weight of the active constituents;
Quercetin in an amount ranging from 0.5% to 8% by weight of the active constituents;
Luteolin in an amount ranging from 1% to 10% by weight of the active constituents;
Baicalin in an amount ranging from 1% to 15% by weight of the active constituents; and
Piperine in an amount ranging from 0.03% to 3% by weight of the active constituents; and (ii) one or more excipients.

6. The formulation as claimed in claim 5, wherein the one or more excipients includes a bulking agent, a solubilizer, a binder, a lubricant, a thickening agent, a flavouring agent, a colouring agent, a tonicity agent, a sweetening agent, a buffering agent, a suspending agent, a preservative, and a solvent.

7. The formulation as claimed in claim 5, wherein the suspension formulation comprises active constituents in an amount ranging from 1% to 20% by weight of the formulation and the rest being one or more excipients.

8. A composition for management of COVID-19 and associated disorders, said composition comprising:
(i) active ingredients comprising: an extract of Citrus reticulata in an amount ranging from about 2% to about 20% by weight of the active ingredients; an extract of Curcuma longa in an amount ranging from about 5% to about 20% by weight of the active ingredients; an extract of Camellia sinensis in an amount ranging from about 5% to about 25% by weight of the active ingredients; an extract of Sophora japonica L. in an amount ranging from about 10% to about 40% by weight of the active ingredients; an extract of Arachis hypogaea in an amount ranging from about 2% to about 20% by weight of the active ingredients; an extract of Oroxylum indicum in an amount ranging from about 20% to about 40% by weight of the active ingredients; and an extract of Piper nigrum L. in an amount ranging from about 0.01% to 3.0% by weight of the active ingredients; and (ii) one or more excipients.

9. The composition as claimed in claim 8, wherein the composition comprises active ingredients in an amount ranging from 1% to 20% by weight of the composition and the rest being one or more excipients.

10. The composition as claimed in claim 8, wherein the composition is formulated as an orally ingestible liquid.
